(19) United States
(12) Patent Application Publication
(10) Pub. No.: US 2026/0041519 A1
(43) Pub. Date: Feb. 12, 2026

(54) CALIBRATING INTRAOCULAR ILLUMINATION

(71) Applicant: Alcon Inc., Fribourg (CH)

(72) Inventors: Paul R. Hallen, Colleyville, TX (US); Andrew Macdonald, Fort Worth, TX (US)

(21) Appl. No.: 19/280,336

(22) Filed: Jul. 25, 2025

Related U.S. Application Data
(60) Provisional application No. 63/681,213, filed on Aug. 9, 2024.

(51) Int. Cl. A61B 90/30

(52) U.S. Cl. CPC A61B 90/30

(57) ABSTRACT

An illumination system includes a light source configured to generate light having a first optical property and a transscleral illumination device configured to transmit light having the first optical property across a sclera and into a vitreous cavity of an eye. A sensor is configured to detect light in the vitreous cavity of the eye as having a second optical property. The sensor generates a first signal indicative of the second optical property. One or more processors receive the first signal and cause the light source to generate light having a third optical property based on a the first signal. The sensor detects light in the vitreous cavity of the eye as having an optical property that is at least within a threshold of the first optical property. The sensor generates a second signal indicative of the first optical property.

### INTRODUCTION

Anatomically, the human eye is divided into two distinct regions: the anterior segment and the posterior segment. The anterior segment includes the lens and extends from the outermost layer of the cornea to the posterior of the lens capsule. The posterior segment of the eye includes the anterior hyaloid membrane and all of the ocular structures behind it, such as the vitreous humor, retina, choroid, and the optic nerve.

Vitreoretinal surgery is performed within the posterior segment of the human eye to treat serious conditions including, but not limited to, age-related macular degeneration (AMD), diabetic vitreous hemorrhage, macular holes, retinal detachment, epiretinal membrane, diabetic retinopathy, and cytomegalovirus retinitis. Such procedures frequently involve the severance and removal of portions of the vitreous humor from the posterior segment of the eye. The vitreous humor is a colorless, gel-like substance made of water, collagen, and hyaluronic acid.

Vitreoretinal surgery may require incisions and insertion of surgical instruments within an eye to repair the retina and/or perform portions of various surgical procedures. In order to visualize the retina and the surgical instruments within the eye, it is important that enough light is available to illuminate the surgical site. It is also important that the light has particular optical properties which can vary for different visualization targets.

For instance, light of a first color (e.g., wavelength) may improve an optical contrast between a first visualization target and its background while light of a second color improves an optical contrast between a second visualization target and its background. During a surgical procedure involving the first visualization target, settings of a light source can be adjusted to match specific settings that are configured to produce the light of the first color. However, the light of the first color transmitted from the light source may be altered in a variety of ways before the light reaches the first visualization target. For example, in a transscleral illumination setting, the sclera acts as a filter such that the light of the first color transmitted from the light source no longer has the first color when the light reaches the first visualization target, which is undesirable. In other words, although a user may select a particular color setting for the illumination light and although the laser source may generate the illumination light with the requested color, the illumination light that is actually observed in the vitreous cavity by the surgeon can have a different color due to the filtering effect the sclera may have on the illumination light as it is transscleraly propagated into the vitreous cavity.

### SUMMARY

Aspects of the present disclosure relate to intraocular illumination and visualization, and more specifically, to calibrating intraocular illumination.

In certain embodiments, an illumination system includes a light source configured to generate light having a first optical property and a transscleral illumination device configured to transmit light having the first optical property across a sclera and into a vitreous cavity of an eye. A sensor is configured to be exposed to the vitreous cavity and detect light in the vitreous cavity of the eye as having a second optical property. The sensor generates a first signal indicative of the second optical property. One or more processors are configured to execute instructions that cause the one or more processors to receive the first signal indicative of the second optical property from the sensor and cause the light source to generate light with a third optical property based on the first signal. Subsequent to the light source generating light with the third optical property, the sensor is configured to detect light in the vitreous cavity as having an optical property that is at least within a threshold of the first optical property and generate a second signal indicative of the first optical property. Subsequent to the one or more processors receiving the second signal, the light source continues to generate light detected as having the optical property that is at least within the threshold of the first optical property.

In certain embodiments, a method includes generating, by a light source, light having a first optical property and transmitting, by a transscleral illumination device, light having the first optical property across a sclera and into a vitreous cavity of an eye. The method further includes detecting, by a sensor, light in the vitreous cavity of the eye as having a second optical property and generating, by the sensor, a first signal indicative of the second optical property. The method further includes receiving, by one or more processors, the first signal indicative of the second optical property and causing, by the one or more processors, the light source to generate light having a third optical property based on the first signal. The method further includes, subsequent to the light source generating light having the third optical property, detecting, by the sensor, light in the vitreous cavity of the eye as having an optical property that is at least within a threshold of the first optical property and generating, by the sensor, a second signal indicative of the first optical property. The method further includes receiving, by the one or more processors, the second signal and, subsequent to the one or more processors receiving the second signal, causing, by the one or more processors, the light source to continue to generate light detected as having the optical property that is at least within the threshold of the first optical property.

### BRIEF DESCRIPTION OF THE DRAWINGS

The drawings described herein are for illustrative purposes only, are schematic in nature, and are intended to be exemplary rather than to limit the scope of the disclosure.

FIG. 1 illustrates a representation of a human eye, according to embodiments described herein.

FIG. 2A illustrates a representation of intraocular light having a first optical property, according to embodiments described herein.

FIG. 2B illustrates a representation of intraocular light having a second optical property, according to embodiments described herein.

FIG. 3A illustrates a representation of light with a first optical property before transmission across a sclera, and detection of the light in a vitreous cavity of the eye as having a second optical property, according to embodiments described herein.

FIG. 3B illustrates a representation of light with a third optical property before transmission across a sclera, and detection of the light in a vitreous cavity of the eye as having an optical property that is at least within a threshold of a first optical property, according to embodiments described herein.

FIGS. 4A and 4B illustrate an example of modifying light generated by a light source to improve an optical contrast between a colored dye and a vitreous cavity of an eye, according to embodiments described herein.

FIGS. 5A and 5B illustrate an example of modifying light generated by a light source to improve an optical contrast between a colored portion of an instrument and a vitreous cavity of an eye, according to embodiments described herein.

FIGS. 6A and 6B illustrate an example of modifying light generated by a light source to improve an optical contrast between the vitreous and a vitreous cavity of an eye, according to embodiments described herein.

FIG. 7A illustrates an example of a surgical system, according to embodiments described herein.

FIG. 7B illustrates an example of an illumination calibration system, according to embodiments described herein.

FIG. 8 illustrates an example method for modifying light generated by a light source, according to embodiments described herein.

The above summary is not intended to represent every possible embodiment or every aspect of the subject disclosure. Rather the foregoing summary is intended to exemplify some of the novel aspects and features disclosed herein. The above features and advantages, and other features and advantages of the subject disclosure, will be readily apparent from the following detailed description of representative embodiments and modes for carrying out the subject disclosure when taken in connection with the accompanying drawings and the appended claims.

### DETAILED DESCRIPTION

Aspects of the present disclosure relate to intraocular visualization, and more specifically, to calibrating intraocular illumination light transmitted from a light source.

The designations “first” and “second” as used herein are not meant to indicate or imply any particular positioning or other characteristic. Rather, when the designations “first” and “second” are used herein, they are used only to distinguish one component from another. The terms “attached,” “connected,” “coupled,” and the like mean attachment, connection, coupling, etc., of one part to another either directly or indirectly through one or more other parts, unless direct or indirect attachment, connection, coupling, etc., is specified.

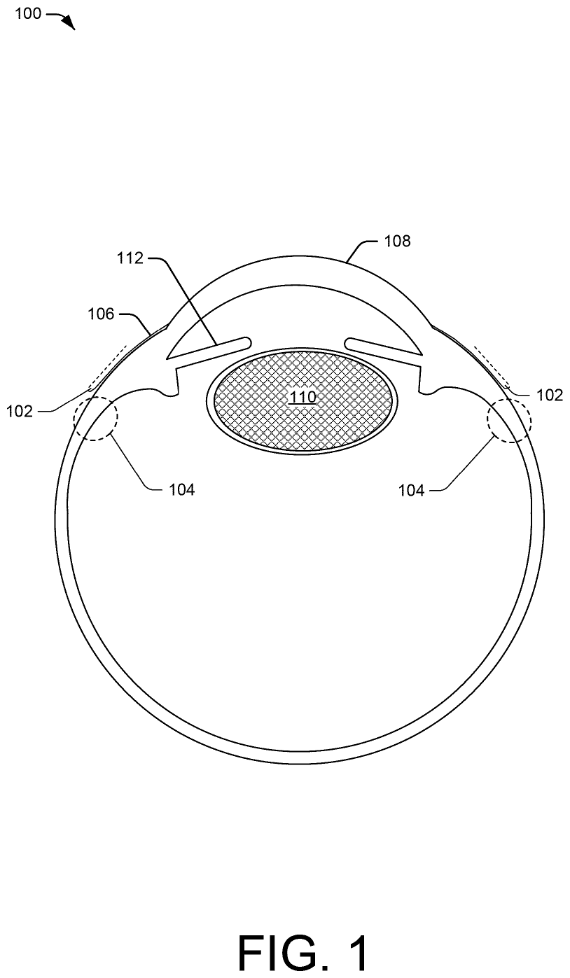

FIG. 1 illustrates a representation of a human eye 100, according to embodiments described herein. As depicted in FIG. 1, the representation illustrates a fornix 102, a pars plana 104, a sclera 106, a cornea 108, a lens 110, and an iris 112. The fornix 102 is a recessed portion of the conjunctiva that is formed where the eyelids interface with the sclera 106.

The pars plana 104 is a region within the ciliary body commonly utilized to access the posterior segment during vitreoretinal surgical procedures (e.g., to remove portions of the vitreous humor). This access is typically achieved via cannulas which are inserted into small incisions made in the pars plana 104 during the procedure. For instance, the cannulas may control an intraocular pressure and/or mitigate trauma to ocular tissue from inserting/removing various surgical instruments.

The sclera 106 is the white/opaque fibrous tissue that is the structural layer of the outer eye and forms its round shape. The sclera 106 extends from the cornea 108 to the optic nerve at the back of the eye 100. The cornea 108 covers the iris 112 which is the colored part of the eye 100 that controls the size of the pupil. The pupil allows light into the eye 100 which the lens 110 focuses on the retina at the back of the eye 100. The retina includes photoreceptor cells which convert the light into signals for visual perception.

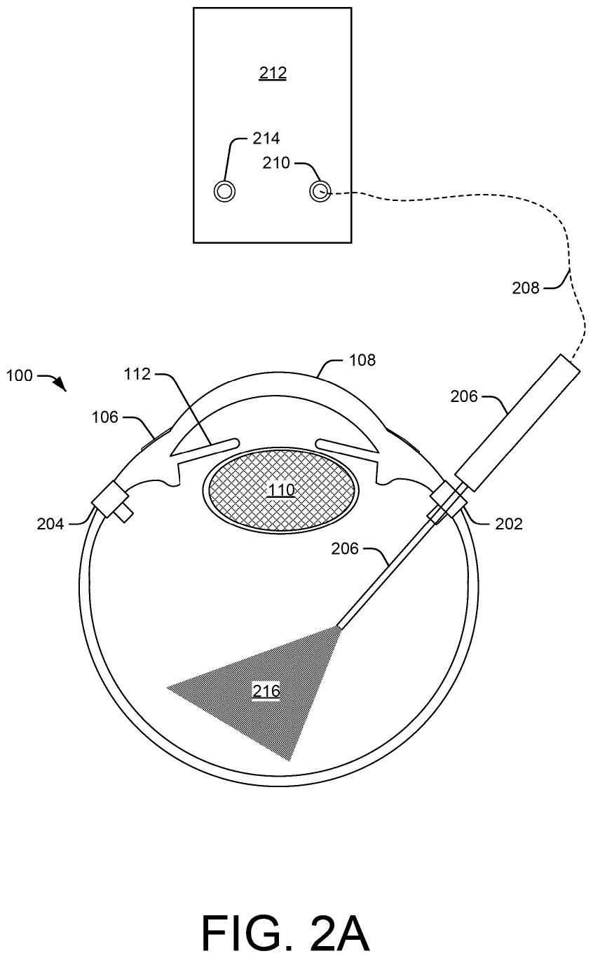

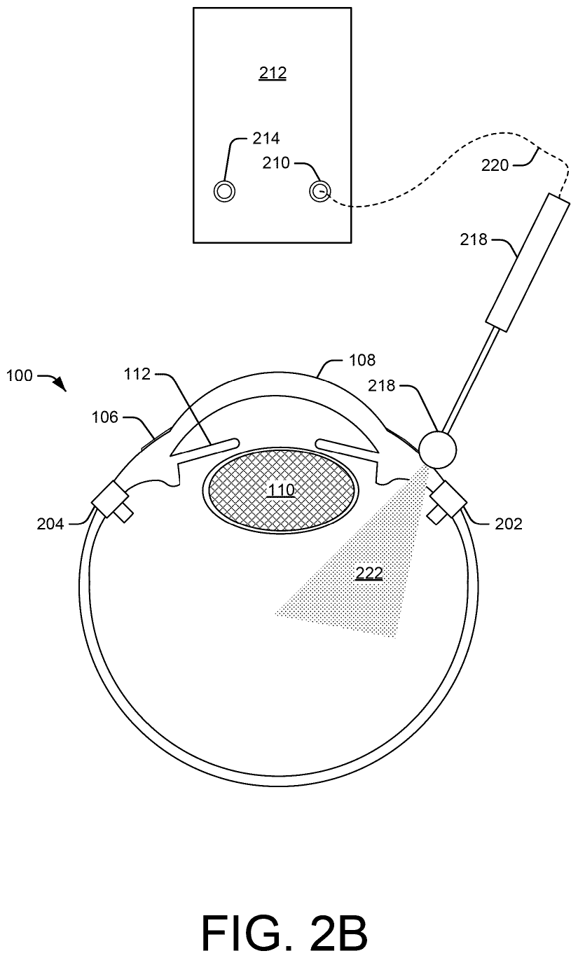

FIG. 2A illustrates a representation of intraocular light having a first optical property according to embodiments described herein. As shown, the representation includes the eye 100. A first cannula 202 and a second cannula 204 have been inserted into incisions made in the pars plana 104. An illumination probe 206 (e.g., a light pipe) is disposed in the first cannula 202. The illumination probe 206 includes an optical fiber 208 which is illustrated to be connected to an illumination port 210 of a light source 212. In some embodiments, the light source 212 includes a spectrophotometer port 214. The light source 212 may be included in an ophthalmic surgical system or the light source 212 can be a standalone light source.

The first optical property may correspond to a first color or hue corresponding to a first set of red-green-blue (RBG) values. In some embodiments, a user specifies the first set of RGB values as input settings to the light source 212, and the light source 212 may be configured to generate light with a first color 216 based on the user-provided settings. Notably, the user may specify other optical properties of light in the form of the input settings to the light source 212 such as an intensity, a color temperature, a direction, a distribution (beam angle), a polarization, a coherence, a spectral composition, a modulation, or another optical property of light which can be adjusted by the light source 212. For example, a user may select a first color or input the first set of RGB values associated with the first color that the user may wish to observe in the vitreous cavity to achieve an expected amount of contrast and visibility. In response, the light source 212 generates light having the first color 216 and transmits the light into the optical fiber 208 via the illumination port 210. As shown, the light is then transmitted through the optical fiber 208, out from a tip of the illumination probe 206, and into a vitreous cavity of the eye 100. In the vitreous cavity, the user or surgeon is then able to observe the desired color and benefit from the visibility and contrast the particular color provides.

In other words, when an illumination probe (e.g., such as illumination probe 206 inserted into the vitreous cavity) is utilized during a procedure, the color observed by the surgeon as the light is being propagated into the vitreous cavity is the same or substantially the same as the color or RGB values that are requested from or inputted as a user request by the user into the light source 212. In particular, when light that is emitted by an illumination device does not travel across the sclera, little to no change occurs to its optical properties (e.g., color). However, as shown in FIG. 2B, when the illumination device is transscleral, one or more optical properties, such as the color, of light that travels across the sclera may change.

FIG. 2B illustrates a representation of intraocular light having a second color 222, according to embodiments described herein. The representation includes a transscleral illuminator 218 configured to transmit light into the vitreous cavity of the eye 100 across the sclera 106. The transscleral illuminator 218 includes an optical fiber 220 which is connected to the illumination port 210 of the light source 212. As described above, as light having the first color 216 may be ideal for visualizing specific anatomical features of the eye 100, the user of the light source 212 may configure the settings of the light source 212 to generate such light.

As such, the light source 212 generates light corresponding to the first set of RGB values and focuses it into the optical fiber 220 via the illumination port 210. The light corresponding to the first set of RGB values is then transmitted through the optical fiber 220, out from a tip of the transscleral illuminator 218, and into the sclera 106. However, as the light is transmitted across the sclera 106, the sclera changes the first color 216 into the second color 222. In other words, the sclera 106 acts as an optical filter which changes the light having the first color 216 into the light having the second color 222. The light having the second color 222, however, may not create the desired contrast and may not be ideal for visualizing the specific anatomical features of the eye 100, which need to be visualized as part of the procedure being performed in the representation.

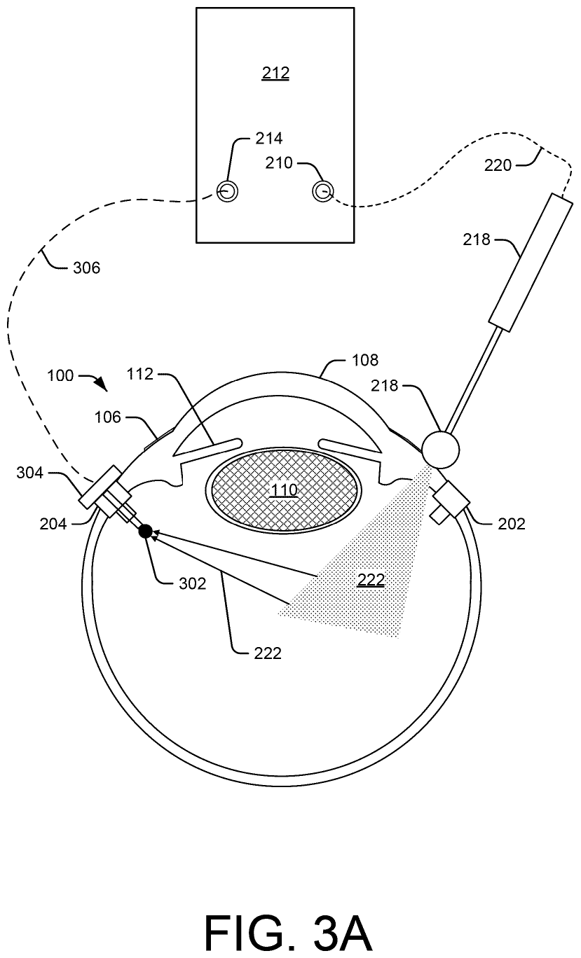

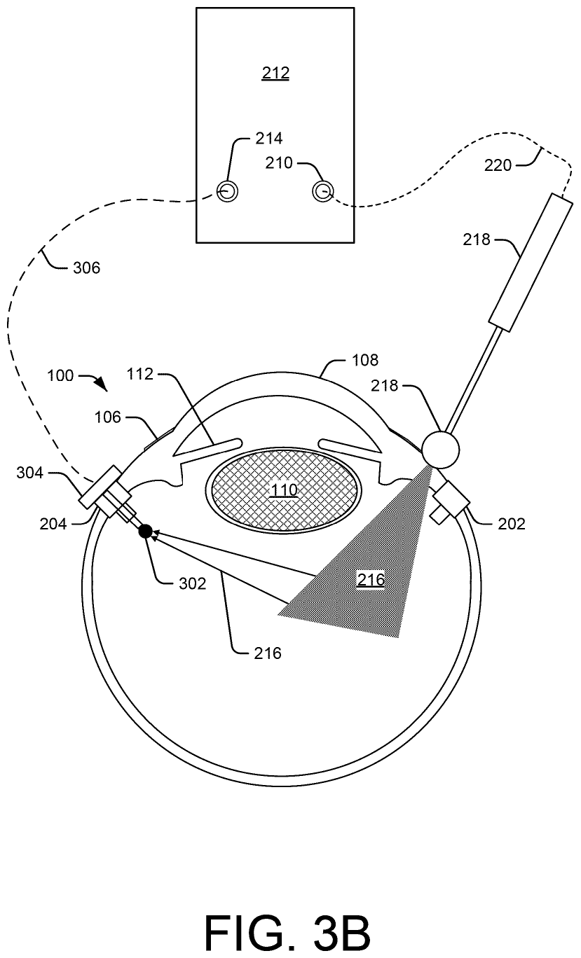

Accordingly, certain embodiments herein provide a system for detecting the second color 222 of the light as propagated in the vitreous cavity and adjusting (e.g., automatically) the settings of the light source 212 in order to achieve the first color 216 in the vitreous cavity. For example, the surgeon may insert a sensor into the patient's eye that detects the second color 222 and generates a signal representative of the second color 222. The sensor then transmits the signal to one or more processors that are configured to cause the light source 212 to adjust its settings in order to generate light that will include the first color 216 when it enters the vitreous cavity. For example, the light source 212 may adjust its settings to generate light with a third color corresponding to a third set of RGB values. As the light having the third color is transmitted across the sclera 106, the sclera filters the light, thereby changing the third color to the first color 216 in the vitreous cavity, as initially desired by the user. FIGS. 3A-3B, 4A-4B, 5A-5B, and 6A-6B provide examples of a system that is configured to detect the optical properties of light being propagated in the vitreous cavity and adjusting the settings of the light source 212 generating the light to achieve a desired optical property.

For example, FIG. 3A illustrates a system for detecting the second color 222 of the light as propagated in the vitreous cavity and adjusting (e.g., automatically) the settings of the light source 212 in order to achieve the first color 216 in the vitreous cavity, as may be desired by the user, according to embodiments described herein. As described above in relation to FIG. 2B, the light source 212 is initially configured, such as by one or more user defined settings, to generate light corresponding to the first set of RGB values and having the first color 216. As such, the light source 212 generates the light corresponding to the first set of RGB values and focuses it into the optical fiber 220 via the illumination port 210. The produced light is then transmitted through the optical fiber 220, out from a tip of the transscleral illuminator 218, and into the sclera 106. As the light associated with the first set of RGB values is transmitted across the sclera 106 and into the vitreous cavity of the eye 100, the first color 216 changes into the second color 222 which corresponds to a second set of RGB values.

As shown, the system includes a sensor 302 that is inserted in the second cannula 204 whose base 304 can secure the sensor 302 to the second cannula 204 during a procedure. Inserting the sensor 302 in the second cannula 204 exposes the sensor 302 to the vitreous cavity of the eye 100. The sensor 302 includes an electrical connection 306 which is coupled to the spectrophotometer port 214 of the light source 212. In some embodiments, the sensor 302 includes a detector of a spectrophotometer configured to measure optical properties of light, such as the color. In some embodiments, the sensor 302 includes an integrating sphere configured to measure optical properties of light.

As shown in FIG. 3A, the sensor 302 detects the light having the second color 222 in the vitreous cavity and generates a signal that is indicative of the second color 222, such as a signal corresponding to the second set of RGB values. The sensor 302 then communicates that signal to the light source 212 via the electrical connection 306. In some embodiments, one or more processors of the light source 212 then execute instructions that cause the light source 212 to modify its settings (e.g., color settings, RGB values, etc.) in order to generate light that can have the first color 216 in the vitreous cavity subsequent to filtering by the sclera 106. Using the adjusted settings, the light source 212 may then generate light with a third color corresponding to a third set of RGB values.

In certain embodiments, modifying settings to achieve a particular optical property, such as a particular color inside the vitreous cavity, may be accomplished in one of a variety of ways. In one example, the one or more processors may be configured to execute one or more functions stored in a memory to, for example, map the RGB values of the second color 222 to the RGB values of the third color. In such an example, the one or more functions can be designed such that the RGB values of the first color 216 can be achieved in the vitreous cavity by causing the light source 212 to generate light corresponding to the RGB values of the third color, based on the sensor detected RGB values of the second color 222.

In some examples, the one or more functions may be rules-based functions designed to map RGB values of the second color 222 to RGB values of the third color in order to achieve the RGB values of the first color 216 in the vitreous cavity. Rules-based functions are based on rules, such as “if and else” rules, that may read as follows: if the light in the vitreous cavity is detected to have the second color 222 corresponding to the second set of RGB values, and the user desires to visualize light with the first color 216 having the first set of RGB values, then generate light with a third set of RGB values corresponding to the third color.

In some other examples, the one or more functions may be representative of a machine learning model that has been trained to take, as input, RGB values of the second color 222 as detected by the sensor, and output RGB values of the third color in order to achieve the first color 216 in the vitreous cavity.

Additionally or alternatively, any variety of optimization techniques (e.g., gradient descent or other algorithms) known to one of ordinary skill in the art may be used to minimize the difference between the RGB values detected by the sensor 302 and the user-desired RGB values corresponding to the first color 216.

Further, in some examples, modifying settings to achieve a particular optical property may be an iterative process. For example, the sensor 302 may continuously detect the RGB values of light that is being propagated in the vitreous cavity and, with each iteration, the light source 212 may accordingly adjust the RGB values with which light is being propagated until the sensor detected RGB values match or are within a defined threshold of the user-desired RGB values. In some embodiments, after the sensor detects the first color 216, the light source 212 or the surgical console that includes or is coupled to the light source 212 may alert the surgeon that the desired RGB values have been achieved, in which case the surgeon may take the sensor 302 out of the cannula 204.

In some other embodiments, even after achieving the user-desired color, the sensor 302 may remain in the cannula 204 to continuously or periodically detect light within the vitreous cavity of the eye 100 to determine whether the color of the light deviates from the user-desired color. If the sensor-detected RGB values reflect a deviation from the user-desired RGB values, then the one or more processors of the light source 212 execute instructions to cause the light source 212 to modify light in order to maintain the first color 216 within the vitreous cavity of the eye 100. For example, moving the tip of the transscleral illuminator 218 to depress a different portion of the sclera 106 may alter the first color 216. In this example, the sensor 302 detects different color and communicates the corresponding RGB values to the light source 212. The one or more processors of the light source 212 then execute instructions which cause the light source 212 to adjust RGB values of the light to maintain the first color 216 within the vitreous cavity of the eye 100.

FIG. 3B illustrates a representation of light in the vitreous cavity of the eye 100 as having the first color 216, according to embodiments described herein. This light is generated outside of the vitreous cavity with the third color and then transmitted from the light source 212 into the optical fiber 220 via the illumination port 210, out from the tip of the transscleral illuminator 218, and into the sclera 106. As the light having the third color is transmitted across the sclera 106, the sclera filters the light's color into the first color 216 or into a color that is at least within a threshold of the first color 216. The threshold may be defined in one of a variety of ways. For example, the threshold can be based on human visual perception such that the threshold corresponds to a difference between the particular color and the first color 216 which can be detected and quantified by the sensor 302 but that is not visually distinguishable by humans. In another example, the threshold may be defined based on distances between RGB color values of the first color and RGB color values of the particular color. In some embodiments, the threshold can be specified by the user of the light source 212 as a configurable setting of the light source 212. For example, the sensor 302 may generate a signal indicative of the first color 216 if the sensor 302 detects light in the vitreous cavity as having the first color 216 or if the sensor 302 detects light in the vitreous cavity as having the particular color that is at least within the threshold of the first color 216.

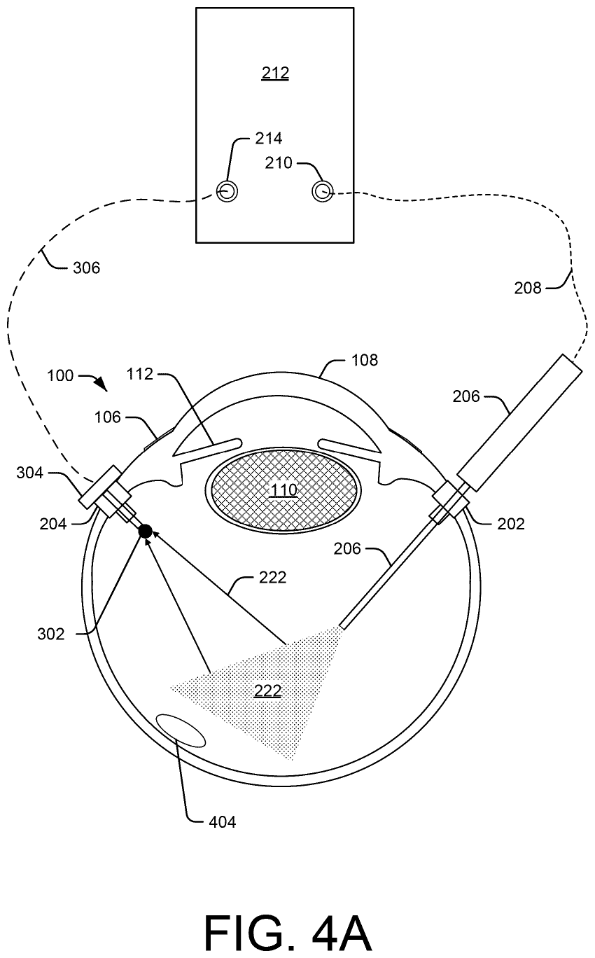

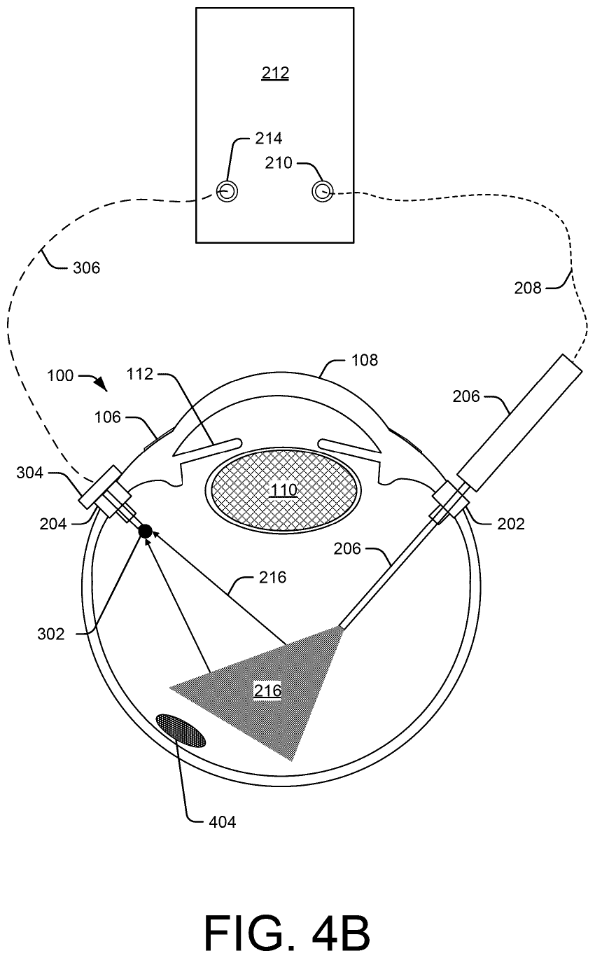

FIGS. 4A and 4B illustrate an example of modifying light transmitted from a light source 212 to improve the optical contrast between a colored dye and a vitreous cavity of an eye 100, according to embodiments described herein. For example, FIG. 4A illustrates a representation of light with the second color 222, which creates a low contrast when illuminating a colored dye 404. FIG. 4B illustrates a representation of light in the vitreous cavity with the first color 216, which creates the desired high contrast when illuminating colored dye 404. The modification of the light with the second color 222 in the vitreous cavity to light with the first color 216 in the vitreous cavity is performed in a manner similar to what was described in relation to FIGS. 3A and 3B.

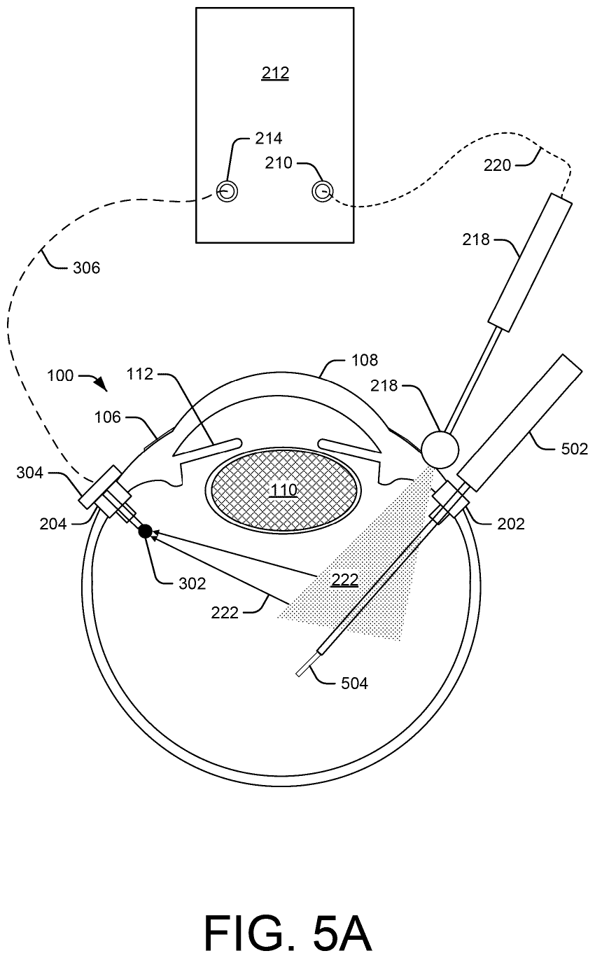

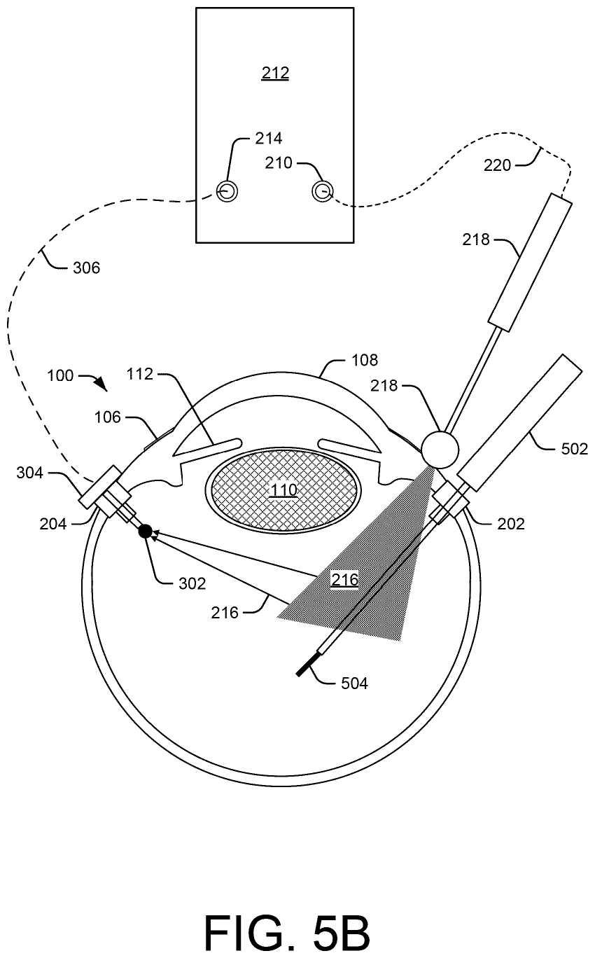

FIGS. 5A and 5B illustrate an example of modifying light transmitted from a light source 212 to improve an optical contrast between a colored portion of an instrument and a vitreous cavity of an eye 100, according to embodiments described herein. FIG. 5A illustrates a representation of light with the second color 222, which creates a low contrast when illuminating a colored tip of the instrument 502. FIG. 5B illustrates a representation of light with the first color 216, which creates the desired high contrast when illuminating the colored tip of the instrument 502. The modification of the light with the second color 222 in the vitreous cavity to light with the first color 216 in the vitreous cavity is performed in a manner similar to what was described in relation to FIGS. 3A and 3B.

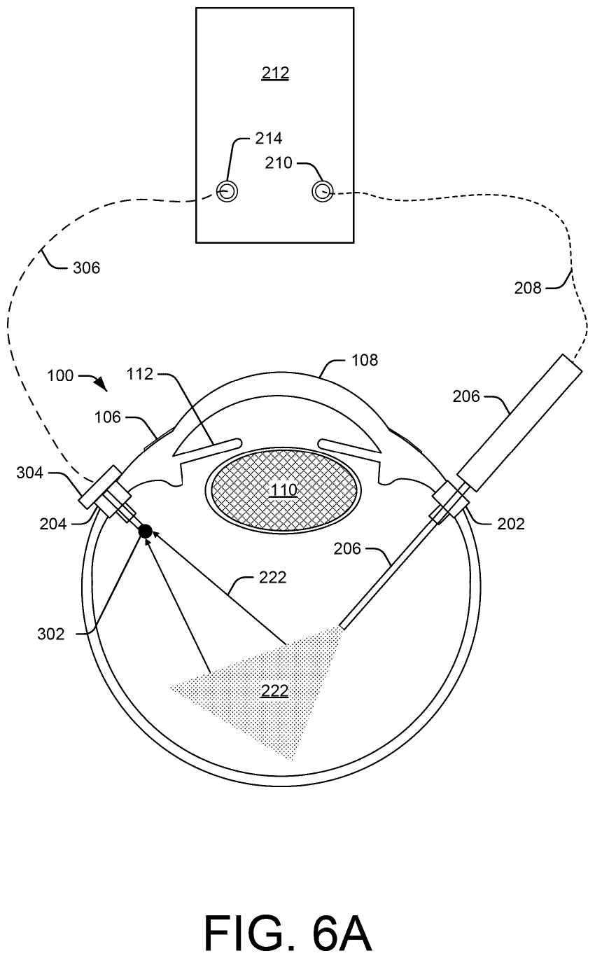

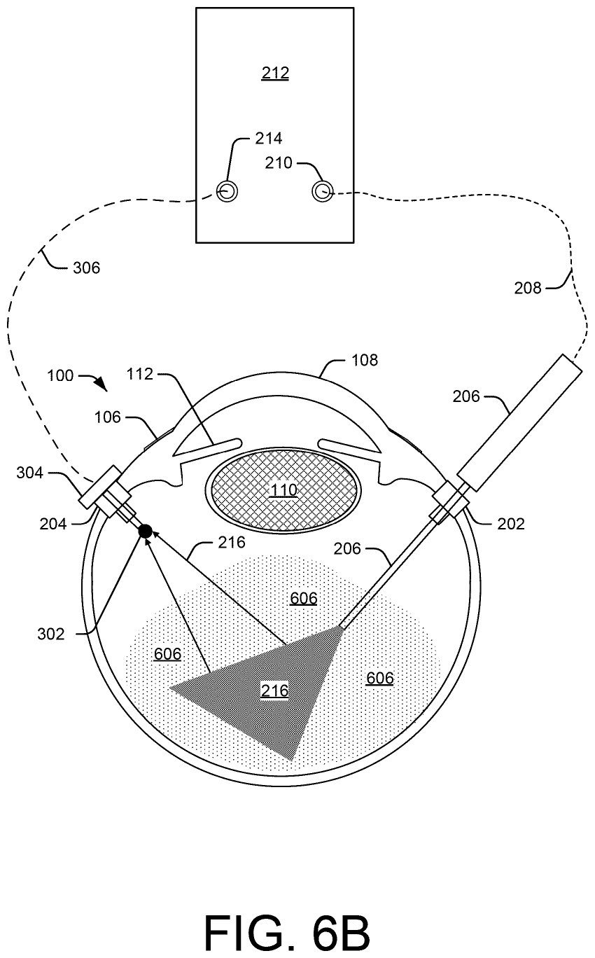

FIGS. 6A and 6B illustrate an example of modifying light transmitted from a light source 212 to improve an optical contrast between the vitreous and the vitreous cavity of an eye, according to embodiments described herein. FIG. 6A illustrates a representation of light that is propagated in the vitreous cavity with the second color 222.

FIG. 6B illustrates a representation of light having the first color 216 in the vitreous cavity, which creates an improved optical contrast between the vitreous and the vitreous cavity of the eye 100.

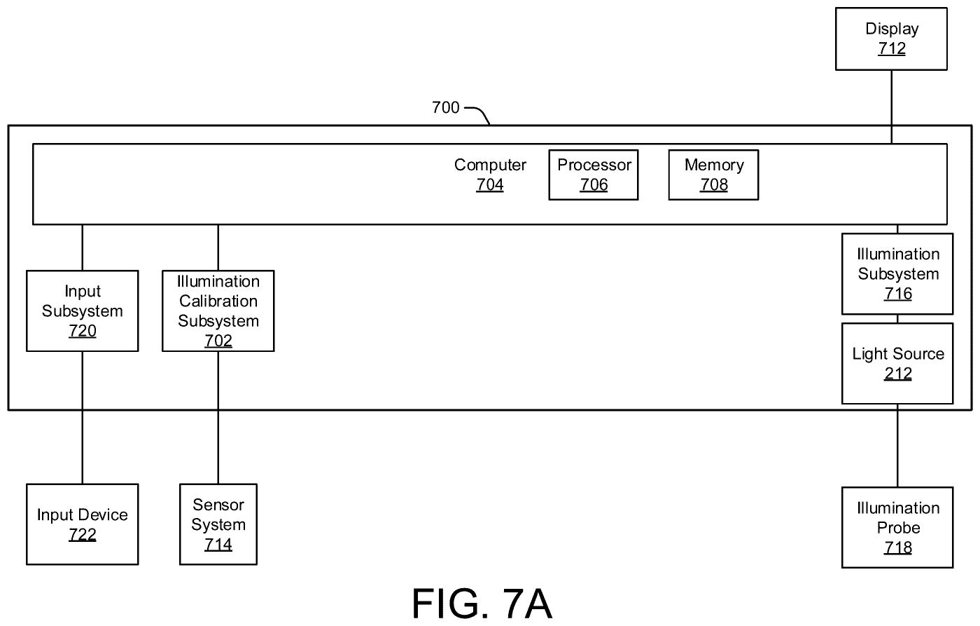

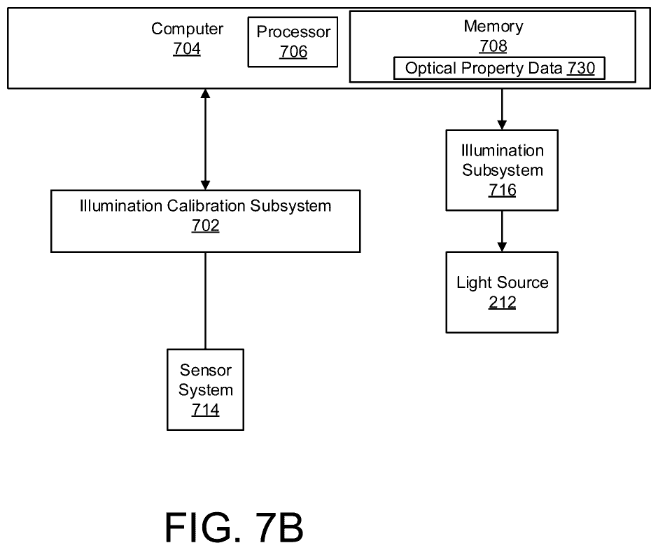

FIG. 7A illustrates an example of a surgical system 700, according to embodiments described herein. In some embodiments, the surgical system 700 includes an ophthalmic surgical console for performing ophthalmic surgical procedures. The surgical system 700 includes an illumination calibration subsystem 702 which is coupled to a computer 704. The computer 704 includes a processor 706 and a memory 708. In some embodiments, a display device 712 is coupled to the computer 704. The display device 712 can include a user interface configured to receive user inputs such as inputs specifying optical properties of light or inputs requesting that one or more optical settings used to produce a particular light output are captured in order to reproduce the particular light output.

The illumination calibration subsystem 702 is coupled to a sensor system 714 which includes the sensor 302. In some embodiments, the sensor system 714 can include multiple sensors in addition or alternative to the sensor 302. The surgical system 700 includes an illumination subsystem 716 which may include the light source 212. The illumination subsystem 716 is coupled to the computer 704 and also coupled to an illumination probe 718 via the light source 212. In some embodiments, the illumination probe 718 can include the illumination probe 206, the transscleral illuminator 218, and/or other types of illumination probes. An input subsystem 720 is coupled to the computer 704 and an input device 722 such as a footswitch.

FIG. 7B illustrates an example of an illumination calibration system, according to embodiments described herein. The illumination calibration system includes the illumination calibration subsystem 702, the computer 704, and the sensor system 714. In some embodiments, the memory 708 of the computer 704 includes optical property data 730. In some embodiments, the optical property data 730 can describe how to adjust light output from the light source 212 based on measured properties of the light within the vitreous cavity of the eye 100. In some embodiments, the optical property data 730 describes one or more captured optical settings used to produce particular light within the vitreous cavity of the eye 100 in order to reproduce the particular light within the vitreous cavity of the eye 100. For example, the optical property data 730 can describe rules-based functions that map RGB values of an observed color to RGB values of a different color in order to produce light having a desired color. In some embodiments, the optical property data 730 describes at least one user specified property of light and corresponding settings of the light source 212 that are configured to produce light having the at least one user specified property. In some embodiments, the optical property data 730 describes a particular hue of light and corresponding settings of the light source 212 that are configured to produce light having the particular hue. In some embodiments, the optical property data 730 describes different optical properties of light and corresponding settings of the light source 212 that are configured to produce light having the different optical properties.

The illumination calibration subsystem 702 receives light properties measured by the sensor 302 within the vitreous cavity of the eye 100 from the sensor system 714. The illumination calibration subsystem 702 communicates the light properties to the computer 704, and the computer 704 implements the processor 706 to execute instructions that cause the processor 706 to control the illumination subsystem 716 to modify light transmitted from the light source 212 based on the light properties. The sensor 302 measures additional light properties of the modified light transmitted from the light source 212, and the illumination calibration subsystem 702 receives the additional light properties from the sensor system 714. The illumination calibration subsystem 702 communicates the additional light properties to the computer 704. In some embodiments, the computer 704 implements the processor 706 to execute instructions that cause the illumination subsystem 716 to modify light transmitted from the light source 212 based on the additional light properties.

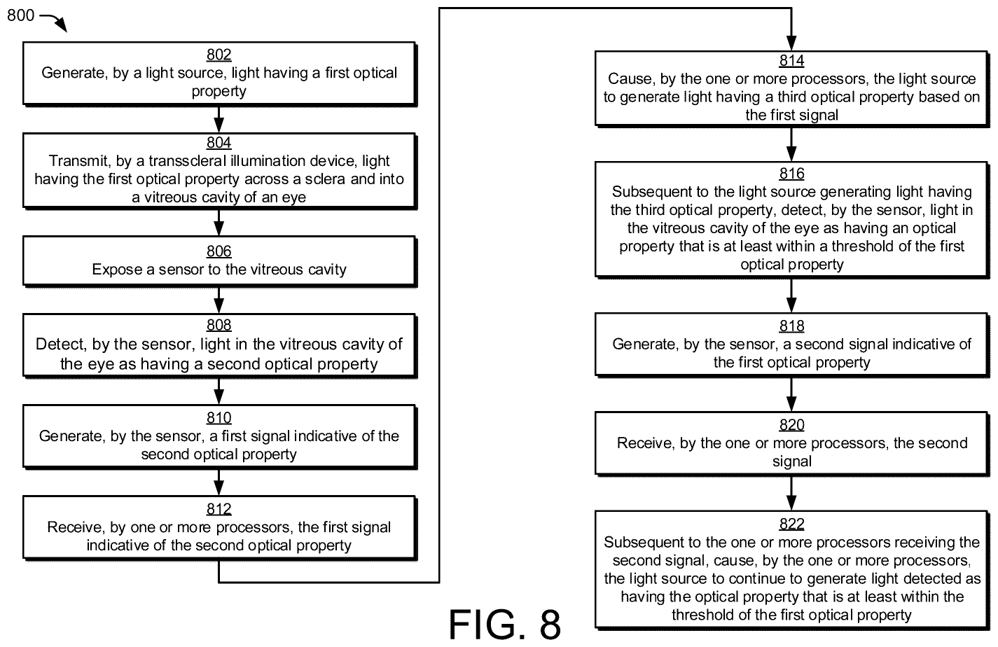

FIG. 8 illustrates an example method 800 for modifying light generated by a light source, according to embodiments described herein. At operation 802, light is generated by a light source (e.g., light source 212) having a first optical property. At operation 804, light having the first optical property is transmitted by a transscleral illumination device (e.g., transscleral illuminator 218) across a sclera and into a vitreous cavity of an eye.

At operation 806, a sensor (e.g., sensor 302) is exposed to the vitreous cavity. At operation 808, light in the vitreous cavity of the eye is detected by the sensor as having a second optical property. At operation 810, a first signal is generated by the sensor indicative of the second optical property.

At operation 812, the first signal indicative of the second optical property is received by one or more processors (e.g., processor 706). At operation 814, the one or more processors cause the light source to generate light having a third optical property based on the first signal.

At operation 816, subsequent to the light source generating light having the third optical property, light in the vitreous cavity of the eye is detected by the sensor as having an optical property that is at least within a threshold of the first optical property. At operation 818, the sensor generates a second signal indicative of the first optical property.

At operation 820, the second signal is received by the one or more processors. At operation 822, subsequent to the one or more processors receiving the second signal, the one or more processors cause the light source to continue to generate light detected as having the optical property that is at least within the threshold of the first optical property.

Note that in some embodiments, the process of modifying settings of the light source to achieve a user desired optical property is an iterative process. For example, in some embodiments, subsequent to transmission of light having the first optical property across the sclera and into the vitreous cavity of the eye, the sensor may detect light in the vitreous cavity of the eye as having a fourth optical property and then generate a third signal indicative of the fourth optical property.

The one or more processors then receive the third signal indicative of the fourth optical property from the sensor; and cause the light source to generate light with a fifth optical property based on the third signal, wherein light with the fifth optical property is detected as having the second optical property in the vitreous cavity, and the first signal is generated by the sensor in response to detecting light as having the second optical property in the vitreous cavity.

The disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. An illumination system comprising:
a light source configured to generate light having a first optical property;
a transscleral illumination device configured to transmit light having the first optical property across a sclera and into a vitreous cavity of an eye;
a sensor configured to:
be exposed to the vitreous cavity,
detect light in the vitreous cavity of the eye as having a second optical property, and
generate a first signal indicative of the second optical property; and
one or more processors configured to execute instructions that cause the one or more processors to:
receive the first signal indicative of the second optical property from the sensor;
cause the light source to generate light with a third optical property based on the first signal, wherein:
subsequent to the light source generating light with the third optical property, the sensor is configured to:
detect light in the vitreous cavity of the eye as having an optical property that is at least within a threshold of the first optical property,
generate a second signal indicative of the first optical property,
the light source continues to generate light detected as having the optical property that is at least within the threshold of the first optical property, subsequent to the one or more processors receiving the second signal.

2. The illumination system of claim 1, wherein the sensor is configured to:
subsequent to transmission of light having the first optical property across the sclera and into the vitreous cavity of the eye, detect light in the vitreous cavity of the eye as having a fourth optical property; and
generate a third signal indicative of the fourth optical property.

3. The illumination system of claim 2, wherein the one or more processors are configured to execute instructions that cause the one or more processors to:
receive the third signal indicative of the fourth optical property from the sensor; and
cause the light source to generate light with a fifth optical property based on the third signal, wherein:
light with the fifth optical property is detected as having the second optical property in the vitreous cavity, and
the first signal is generated by the sensor in response to detecting light as having the second optical property in the vitreous cavity.

4. The illumination system of claim 1, wherein the sensor comprises a detector of a spectrophotometer.

5. The illumination system of claim 1, wherein the first optical property is a user specified property.

6. The illumination system of claim 5, wherein the light source generates light with the first optical property in response to receiving a user request through a user interface.

7. The illumination system of claim 1, wherein light with the second optical property in the vitreous cavity corresponds to light that is generated with the first optical property and transmitted across the sclera.

8. The illumination system of claim 1, wherein the first optical property corresponds to a red-green-blue (RGB) value.

9. A method comprising:
generating, by a light source, light having a first optical property;
transmitting, by a transscleral illumination device, light having the first optical property across a sclera and into a vitreous cavity of an eye;
detecting, by a sensor, light in the vitreous cavity of the eye as having a second optical property;
generating, by the sensor, a first signal indicative of the second optical property;
receiving, by one or more processors, the first signal indicative of the second optical property;
causing, by the one or more processors, the light source to generate light having a third optical property based on the first signal;
subsequent to the light source generating light having the third optical property, detecting, by the sensor, light in the vitreous cavity of the eye as having an optical property that is at least within a threshold of the first optical property;
generating, by the sensor, a second signal indicative of the first optical property;
receiving, by the one or more processors, the second signal; and
subsequent to the one or more processors receiving the second signal, causing, by the one or more processors, the light source to continue to generate light detected as having the optical property that is at least within the threshold of the first optical property.

10. The method of claim 9, further comprising:
detecting, by the sensor, light in the vitreous cavity of the eye as having a fourth optical property; and
generating, by the sensor, a third signal indicative of the fourth optical property.

11. The method of claim 10, further comprising:
receiving, by the one or more processors, the third signal indicative of the fourth optical property; and
causing, by the one or more processors, the light source to generate light with a fifth optical property based on the third signal, wherein:
light with the fifth optical property is detected as having the second optical property in the vitreous cavity, and
the first signal is generated by the sensor in response to detecting light having the second optical property in the vitreous cavity.

12. The method of claim 9, wherein the sensor comprises a detector of a spectrophotometer.

13. The method of claim 9, wherein the first optical property is a user specified property.

14. The method of claim 13, wherein the light source generates light with the first optical property in response to receiving a user request through a user interface.

15. The method of claim 9, wherein the first optical property corresponds to a red-green-blue (RGB) value.
